(19) United States
(12) Patent Application Publication
(10) Pub. No.: US 2004/0186359 A1
(43) Pub. Date: Sep. 23, 2004

(54) Afinity biosensor for monitoring biological process

(76) Inventors: Stephen P. Beaudoin, West Lafayette, IN (US); Karl S. Booksh, Gilbert, AL (US); Philip A. Khairallah, Scottsdale, AZ (US); Hassan Loutfi, Scottsdale, AZ (US); Alyssa Panitch, Highley, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/483,586

(22) PCT Filed: Jul. 9, 2002
(86) PCT No.: PCT/US02/23300

(30) Foreign Application Priority Data
Jul. 9, 2001 (US) 60303956

(51) Int. Cl. A61B 6/00

(52) U.S. Cl. 600/310; 600/476; 356/39

(57) ABSTRACT

An optical biosensor carries one or more affinity legends or binding members that bind specifically to a marker being monitored. Light directed along optic fibers illuminates a surface plasmon resonance (SPRN) probe surface on which is immobilized the binding member. A spectrophotometer receives light reflected back along the fiber optic path and provides wavelength information indicative of the absence or presence of surface plasmon resonance indicative of the bound marker in known SPR manner. The probe is used in vitro or in vivo. When used in vivo the fiberoptic light path comprises a catheter that directs the probe to an implant site. For in vivo implantation a housing houses the probe at the implant site and is adapted to filter out larger particles that would adversely affect with the spectral analysis. In one embodiment the probe has two regions on its surface. The first region has no immobilized binding member. The second region does have the binding member immobilized on it. Light returned from the first and second regions can be compared. The presence or absence of a marker bound by the binding member on the second surface is apparent in the similarity or dissimilarity of the spectral information returned from the two regions. The probe can monitor blood, spinal fluid, mucus membrane, wound tissue, implanted organs, urine and other substances for the presence of a marker which may be indicative of a medical condition in an animal or human subject.

### Cross Reference to Related Patent Applications

This application is the National Stage of International Application No. PCT/US02/23300, filed on Jul. 9, 2002, which claims the benefit of U.S. Provisional Application 60/303,956, filed Jul. 9, 2001.

### FIELD OF THE INVENTION

This invention relates to fiber-optic-based, implantable biosensors for in vivo and in vitro monitoring of proteins and other biologically relevant markers that are of clinical use in detecting medical conditions. The biosensor comprises one or more affinity ligands that bind specifically to the marker being observed. Methods for using the biosensors for continuous in vivo or in vitro assays are given. A housing for the biosensor is provided for screening cells and other particulate components of body fluids. In an important aspect of the invention, a method is given for the instant in vivo detection and monitoring of the onset of ischemia and myocardial infarction. Methods for monitoring wound healing are also disclosed.

### BACKGROUND OF THE INVENTION

There is a need for implantable biosensors that yield in vivo, real-time, continuous analyses for biologically relevant markers useful for medical diagnosis; assessment of imminent risk of organ failure, injury or rejection; disease detection/progression; monitoring of therapy and discovery of important components of biological systems. Both in vivo and in vitro sensing are desired.

Of special importance is the need for a biosensor for the in vivo detection and prevention of myocardial infarction. Cardiac disease is among the leading causes of death in the United States. Methods that would allow fast, definitive diagnosis of infarction or ischemia would improve patient care. Currently, patients go to the hospital after experiencing chest pain, and tests are performed to detect cardiac muscle damage. The tests involve electrical monitoring of heart rhythm and the analysis of blood samples to detect markers for cardiac damage, such as creatinine kinase and cardiac troponin T (cTnT). If cardiac damage is found, then antithrombolytic agents are administered to clear the heart blockage, or a catheterization is performed to open the blocked vessel. In the case of patients who have experienced ischemic events without significant damage to the heart, catheterization may or may not be used to increase the opening in the affected vessel. There are several fundamental limitations to this approach. First, there are large classes of patients who experience silent infarctions and ischemic events, including dialysis patients and diabetics. For these individuals, who are generally at high risk for cardiac disease, it is nearly impossible to detect and treat cardiac events. Cardiac disease is the leading killer of such individuals. An implantable sensor that could monitor these patients continuously and signal an alarm as soon as possible after the onset of ischemia or infarction would be of great utility. Second, many patients enter the hospital with unstable angina or other symptoms of ischemia or mild infarction but do not present adequate markers to allow a definitive diagnosis. These patients commonly will have severe infarctions closely after the onset of the initial unstable angina. A way to monitor these patients will allow for intervention therapies to prevent infarction from occurring.

It has been hypothesized that cracks in arterial plaques induce an inflammatory response, including the release of C-reactive protein (CRP). The resulting clot may trigger ischemia or infarction, usually within 40-60 days following the initial crack formation. An implantable sensor to detect the presence of these components in at-risk patients would allow for preventative measures to be pursued before significant cardiac damage occurs.

To achieve the goal of sensitive in situ monitoring of biological processes, a biosensor must be selective to the target marker (protein or class of proteins for research discovery, or other biological markers such as sugars, integrins, nucleic acids, or peptides), sensitive to ˜ng/ml of analyte in vivo or in vitro, of a size sufficiently small to fit in blood vessels for in vivo sensing in the bloodstream, and be constructed of biologically compatible materials. Fiber optic surface plasmon resonance (SPR) sensors have the potential to meet all of these criteria.

Surface plasmon resonance (SPR) spectroscopy has been employed for quantitative and qualitative analysis in analytical chemistry [1, 2, 3], biochemistry [4, 4, 6, 7], physics [8, 9] and engineering [10, 11, 12, 13] applications. SPR sensor technology has become a leading technology in the field of direct real-time observation of biomolecular interactions.

SPR is sensitive to minute refractive index changes at a metal-dielectric surface. Because it is a surface technique that is sensitive to changes of 10⁻⁵ to 10⁻⁶ refractive index (RI) units within approximately 200 nm of the SPR sensor/sample interface, SPR spectroscopy is becoming increasingly popular for monitoring the growth of thin organic films deposited on the sensing layer [14, 15, 16, 17]. As little as 0.01 nm of average film deposition can be detected when the RI difference between the film and bulk solution is 0.1 RI units [14]. Thus, a sub-monolayer of adsorbed protein-like substance (RI=1.4) from an aqueous solution (RI=1.3) can easily be observed.

However, in its simplest form, SPR is not analyte-specific, so that any analyte bound to the surface will induce an SPR signal. This characteristic has limited the usefulness of SPR techniques for monitoring biological processes on a continuous basis in vivo. In the effort to confer specificity on SPR methods, both direct and competitive binding bioassays have been developed for several binding pairs. In these bioassays, the binding of target analytes to specific ligands immobilized on the metal surface triggers an SPR signal [14, 18, 19, 20, 21] that is read with a wave guide technique. The sensitivity of the in vitro bioassays depends on the binding constant of the receptor-ligand system. For the detection of the biological markers of myocardial infarction, namely, cTnT, CRP, CK-MB, or myoglobin, sensitivity of assays must be sufficient to detect the typical infarction-induced concentrations in the body which are on the order of: cTnT 0.15-0.5 ng/ml; CRP 0.1-3.0 mg/L; CK-MB 0-4.3 ng/ml; myoglobin 15-30 ng/ml. However, for in vivo continuous real-time monitoring of markers in biological fluids, sensitivity of assays based on binding pairs is affected by non-specific binding of natural components in biological fluids.

Employing SPR sensing on multimode optical fibers presents distinct advantages for in situ analysis of pharmacological analytes, proteins, and other markers. Combining the sensitivity of SPR analysis with the selectivity of antibodies or other specific receptors yields a powerful sensor system. SPR is a surface technique so the opacity of the blood matrix or biological fluid has minimal effect on the detection limits of the sensor. The response time is fast. For example, blood analgesic levels can be determined within one minute. Since detection limits with SPR are not power-dependent, low-power light sources and detectors can be employed to minimize size and power requirements of the sensor system. The fiber sensor can be made quite small (<200 μm in diameter) such that the sensor can be incorporated into catheters without hampering the performance of the catheter, sensor, or vein. The sensor itself is reusable and capable of withstanding the sterilization environment of an autoclave or UV radiation. The analyte-specific layer is renewable with commercially available products for target applications. It is expected that the breadth of commercially available antibody binding kits will expand throughout the foreseeable future as will the availability of mRNA aptomers and molecularly imprinted polymers as alternative biospecific sensing layers.

Biosensors incorporating SPR surface techniques for monitoring in vivo biomarkers with high specificity and sensitivity have been sought.

### SUMMARY OF THE INVENTION

An implantable biosensor for the in vivo observation of a biological marker in a tissue in an individual has been discovered. The biosensor comprises a surface plasmon resonance (hereinafter termed “SPR”) probe surface having immobilized thereon a binding member capable of binding specifically to the biological marker being observed. The biosensor also comprises means for receiving signals from the implanted probe surface. Preferably a spectrophotometer is provided for measuring the wavelength of the minimum light intensity received from the probe. Means are provided for receiving a first signal from the probe surface after the biosensor is implanted (in vivo testing) or immersed in drawn blood or biofluid (in vitro sensing) and means for receiving a second signal from the probe surface after in the presence and absence of binding of the biological marker to the probe surface in situ. In preferred embodiments of the invention, receiving means comprise two regions on the sensing fiber. In these embodiments, the first region does not have surface immobilized binding member (receives first signal) and the second region does have surface immobilized binding. Means are also provided for comparing properties of the first received signal and the second received signal to determine the presence of the biological molecule. Generally the fluid to be monitored is selected from the group comprising blood, urine, cerebrospinal fluid, mucous membrane, wound tissue and its associated fluid and implanted organs.

In certain preferred embodiments of the invention the biosensor comprises multimode optical fibers. In other preferred embodiments the biosensor comprises a self-referencing optical sensor. The self-referencing sensor may comprise spatially separated sensing areas. The self-referencing sensor may comprise a beveled tip.

In an important aspect of the present invention, the biosensor comprises a housing for the probe surface. The housing is capable of excluding particulate components of the tissue from contact with the probe surface. This exclusion prevents non-specific binding of particulate components and thus achieves a sensitivity of SPR in vivo binding assay hitherto unknown.

In the present invention, the biomolecule being observed is one member of a binding pair and the biosensor comprises a probe surface on which is immobilized the other member of the binding pair. Preferably the binding pairs are members of the group comprising antigen and antibody binding pairs wherein the antigen is a protein, peptide, carbohydrate, drug, or other chemical compound, nucleotide and anti-nucleotide binding pairs, enzyme and receptor binding pairs, carbohydrate and lectin binding pairs, and pharmacological analytes and polymer binding pairs. Most preferably the binding pairs are antigen and antibody binding pairs and the antigen is selected from the group comprising protein, peptide, carbohydrate, drug or other chemical compound and the antibody is capable of binding specifically and with high affinity to the antigen.

In preferred embodiments of the invention the biological marker to be determined is selected from the group comprising protein, peptide, RNA, DNA and carbohydrate. In preferred embodiments of the invention, the biosensor is capable of detecting myocardial infarction in an individual. In these embodiments, the first member of the binding pair is selected from the group comprising cardiac troponin T (cTnT), cardiac troponin I (cTnI), C-reactive protein (CRP), creatinine kinase myocardial band (CK-MB), and cardiac myoglobin (myoglobin); and said second member of said binding pair is an antibody capable of binding specifically to said first member.

In other preferred embodiments of the invention the biosensor is capable of monitoring the progression of wound healing in a tissue to distinguish between healing and non-healing wounds. In these embodiments the biological marker is selected from the group comprising interleukins, matrix proteolases and other components of non-healing wounds, and the antibody is capable of binding specifically and with high affinity to the biological marker.

In an important aspect of the present invention, a method is provided for detecting a biological molecule in a tissue/fluid matrix in an individual. In the method, the present SPR biosensor is implanted at a selected site in the tissue matrix. For in vitro applications, the probe is placed into the fluid sample to be monitored. A first signal is received from the SPR probe surface after its contact with tissue or fluid. A second signal is received from the probe surface at a time after binding occurs between the binding member immobilized on the probe surface and the molecule to be observed, the biological marker of interest. Preferably the signals are spectrographically received wavelength values at minimum reflectance. In certain embodiments, more than one probe may be placed in contact with the sample to be monitored. In these embodiments, each probe may comprise a binding member for a particular biological marker of interest. In preferred embodiments, two regions on the sensing fiber probe are provided. The first region does not have surface immobilized species that would bind with the analyte of interest. The second region contains surface immobilized species that would bind with the analyte of interest. The signal is received by a spectrophotometer that records the wavelength of the minimum refractive index received from the sample. The difference between signals is calculated and compared to signals received from a comparison standard tissue/matrix containing the biological marker to determine the presence of the biological molecule. The method may be used for quantifying the amount of a biological molecule in vitro or in vivo in an individual by comparing the observed properties of the signals to signals received from a biological solution of the molecule at known concentrations.

The method of the present invention may be used to detect a biological molecule in a tissue/matrix selected from the group comprising blood, spinal fluid, mucous membrane, wound tissue, implanted organs and urine. Methods are given for continuous in situ observation of the biological molecule over a determined time period wherein the biosensor is allowed to remain in situ for said period of time. These methods are especially important for monitoring therapy of a medical condition in which the biological molecule is a marker that changes concentration over a period of time in response to the therapy.

These and other aspects of this invention will become evident upon reference to the following detailed description and attached drawings.

### BRIEF DESCRIPTION OF THE DRAWINGS

FIG. 1 is an illustration of the refractive properties of the SPR probe surface.

FIG. 2 is a schematic illustration of a multimode fiber optic SPR sensor.

FIG. 3(a) is schematic illustration of the SPR biosensor.

FIG. 3(b) is a photographic image of an SPR biosensor. For illustrative purposes, each block in FIG. 3(b) is 5 mm long.

FIG. 4 is an illustration of the configurations of a two zone, self referencing sensor.

FIG. 5 displays SPR spectra from a dual sensing area probe. The first spectral dip occurs from the RI (A) on the probe shaft. The second spectral dip is dependent on the RI at the tapered region (13).

FIG. 6 is an SEM (Scanning Electron Microscope) image (19× magnification) of a template made from SU-8 photoresist on a silicon wafer using photolithography. The posts are ˜25 microns tall and ˜80 microns in diameter.

FIG. 7 is an SEM image of a polydimethylsiloxane (PDMS) film (16× magnification) that has adhered to itself due to treatment with a radiofrequency (rf) oxygen plasma. The treatment conditions were 50 sccm of O₂ at a pressure of 120 mtorr for 10 s at a power of 70 W. Upon contact the edges of the film adhered to each other irreversibly.

FIG. 8 is a graphical illustration of the SPR detection of anti-myoglobin immobilization on gold surface on sensor (creates sensor) and of myoglobin binding with immobilized anti-myoglobin.

FIG. 9 is a schematic illustration of antibody/antigen binding and sensor signal.

FIG. 10 is a schematic illustration of a competitive immunoassay for detection of blood-borne marker molecules. a) The sensor in the absence of free antigen, with an SPR signal indicative of a high RI; b) the sensor exposed to free antigen in the blood, binding of the free antigen is thermodynamically favored compared to BSA-tagged antigen; c) once the BSA-tagged antigen is displaced by free antigen, the RI at the probe surface will decrease, shifting the SPR signal to a lower wavelength.

FIG. 11 is a sensogram for the binding of anti-troponin I and troponin I. The numbers on the graph indicate the steps described for the assay.

FIG. 12 is a sensogram of the assay of troponin I with the SPR biosensor of the present invention.

FIG. 13 is a sensogram of the assay of myoglobin, concentration 500 ng/ml, with the SPR biosensor of the present invention.

FIG. 14 is a sensogram of the assay of myoglobin, concentration 25 ng/ml.

### DETAILS OF THE INVENTION

This invention is directed to in optical fiber biosensor for detecting a biological marker in a fluid matrix of an individual by surface plasmon resonance (SPR) measurements. SPR is used generally for characterization of thin films and for monitoring processes at metal interfaces. SPR is an optical sensor technique that may be utilized with a large variety of optical methods. In the present invention, the SPR sensing technique is used to measure refractive indices (RI) from affinity-based thin films and changes in the RI of the films after reaction with an analyte of interest in situ. The technique has been described by Homola et al. [22], the details of which are herein incorporated by reference.

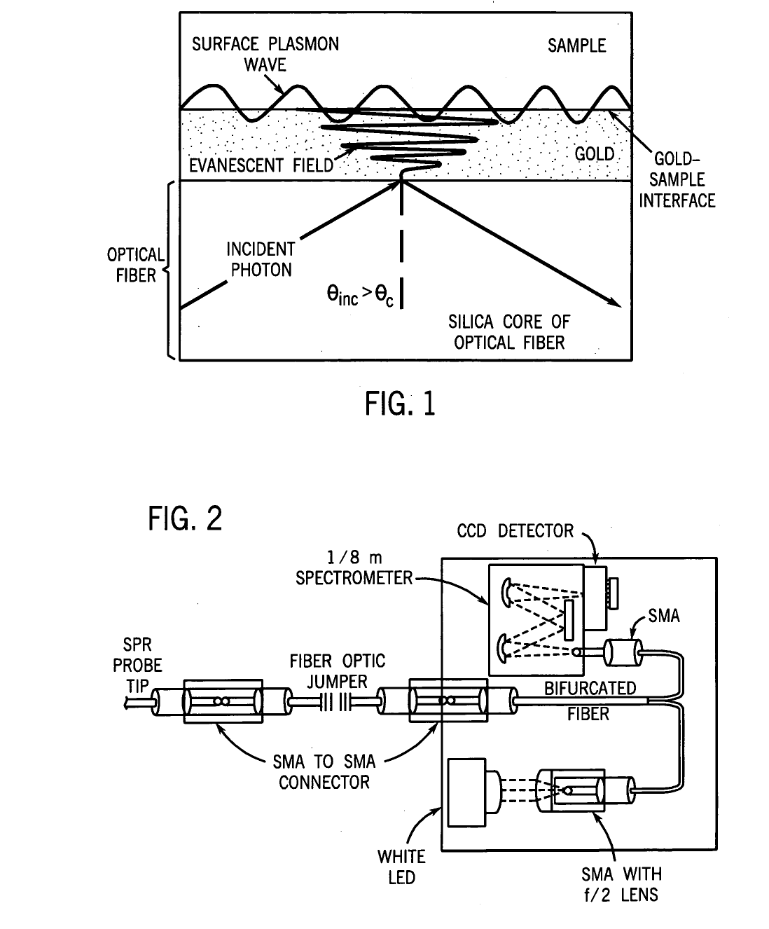

The SPR effect is illustrated schematically in FIG. 1. The photons that excite the surface plasmon wave are completely contained in the optical fiber wave guide. When the photon experiences total internal reflection at the interface of the optical fiber, the evanescent field of the photon extends into the 50 nm thick gold layer. This evanescent field then excites a standing charge density wave of electrons, a surface plasmon wave, along the sensor surface at the gold-sample interface. If the matching conditions are just right, the surface plasmon wave will couple with the sample, and the photon will propagate into the solution. Consequently, photons at exactly the proper wavelength to excite a coupling surface plasmon wave will not continue along the fiber and reflect back to the detector. Thus the refractive index at the gold-sample interface can be correlated to the wavelength of minimal returned light from the sensor.

The advantages derived for employing the SPR technique in an implantable biosensor are threefold: First, SPR spectroscopy can be accurately performed with low light levels. Because the quantitative information is in the wavelength of minimal reflection, not in the intensity of reflection, the intensity of the light does not determine the dynamic range of the sensor. Furthermore, by using low light levels, heating at the fiber tip, such as with a laser, is not a concern. Second, SPR spectroscopy can be performed in very complex, opaque solutions such as encountered in tissues and blood. Because the photons never leave the fiber and the coupling wavelength is insensitive to the absorbance of the sample, SPR spectroscopy can be performed in very complex, opaque solutions. Thus fluctuations in concentration of highly absorbing species such as hemoglobin do not significantly degrade the accuracy or precision of SPR spectroscopy. Thirdly, coating of the sensor to prevent thrombosis does not degrade its utility. Because the photons never leave the optical fiber, the optical transmission is not attenuated when the sensing area is coated with opaque anti-thrombogenic substances.

FIG. 2 is a schematic illustration of the multimode fiber optic SPR sensor of the present invention showing the implantable SPR probe tip and the means whereby the SPR signal is received from the probe tip. A spectrophotometer is illustrated that is suitable means for collecting and processing the sensor data. The SPR signal is in the form of light intensity returned from the sensor as a function of light wavelength. The wavelength of light corresponding to the surface resonance will exhibit a minimum in the returned light spectrum. Calibration of SPR spectra is performed by relating the wavelength of least light return from the sensor to the refractive index (or concentration) of the analyte in solution. This requires accurate and reliable estimation of the minima of normalized spectra. It has been demonstrated that multimode SPR sensors can perform equivalently to planar-prism sensors when multivariate calibration methods are employed [23]. More recently alternative multivariate calibration models have been investigated to determine the best balance between model accuracy and ease of calibration [24]. It has been discovered that the width of the SPR spectra, as collected with multimode fiber sensors, does not impair the ability to accurately and reliably calibrate the sensors when multivariate calibration methods are employed. In preferred embodiments of the invention multimode fiber sensors are employed.

In preferred embodiments of the present invention, the distal end of the fiber optic probe has been modified to shift the dynamic range and increase the sensitivity of the SPR biosensor. With multimode fiber optic sensors, the distribution of angles of light impinging on a sensor surface is determined by the refractive indices of the fiber core and cladding. The desired angle of light is selected by modifying the tip of the fiber. By selectively beveling the distal end of the fiber probe, the wavelength of resonance has been red shifted by more than 100 nm and blue shifted by more than 30 nm. This increases the flexibility of a white-light SPR sensor by increasing the dynamic range of accessible refractive indices and by shifting the resonance to the most sensitive regions of the detector. With the modified tip, sensitivity, measured in wavelength shift per refractive index (RI) change, has been increased by a factor of 6.

The present biosensor detects multiple wavelengths of SPR activity simultaneously on the same probe, thus increasing the information content of a SPR spectrum. The fiber optic SPR sensor of this invention advantageously eliminates the traditional limitation of planar-prism geometry employed with traditional SPR sensors. The present fiber optic biosensor exploits the ‘dual resonance’ feature by coating of the fiber to mitigate the effects that non-analyte dependent sample changes (i.e., sample temperature and density) have on the quantitative capability of SPR sensors.

It is an objective of the present invention to provide a small, inexpensive fiber optic based SPR biosensor for in situ analyses in vivo or in vitro in drawn fluid as for field use. A small, portable SPR sensor system biosensor is provided that employs multimode optical fibers to replace the planar-prism geometry employed with traditional SPR sensors. The fiber optic sensing probes permit reliable analyses in small systems that are inaccessible to other geometries, intravenous analyses, for example. The fiber may be sapphire or silica, preferably silica. The overall footprint and power requirements are sufficiently small to permit field use of the instrument. In certain embodiments the physician/patient can carry the instrument on his belt. FIG. 2 illustrates an instrument (sensor and signal processing). The dimensions of this instrument are approximately 9″×6″×3″, about the inner dimensions of a cigar box.

The biosensor for intravenous purposes must be small enough to be mounted on a catheter for insertion into the circulatory system or other chosen tissue site. In these applications, the biosensor comprises a single fiber optic cable traced along the catheter to receive and transport the signal out of the body. The diameter of the fiber optic cable is preferably between about 200 μm to 50 μm.

A small, low power spectrophotometer accompanies the sensor. For miniaturization purposes, the spectrophotometer may be micromachined on a silicon chip with an embedded light source and array of silicon photo diode detectors. Bench top spectrophotometers commercially available may be used. A ‘minimal’ spectrometer that is optimized for size, weight, and power consumption is provided.

The SPR is constructed to be a small-footprint, low-weight system. A white light emitting diode (LED) provides a stable, low power source of sufficient intensity to easily perform SPR measurements. In certain embodiments, the LED may be battery-powered. The white LED is stable for more than 100 hours of continuous use with a 9V household battery. One leg of a bifurcated silica-silica fiber carries the light to the SPR probe tip. The bifurcated fiber terminates into a sub-miniature version A (SMA) type connector that permits easy attachment of the probe/syringe sampling system to the SPR spectrometer. Reflected light from the sensor tip is analyzed by a spectrometer at the end of the second leg of the bifurcated fiber. The high resolution data is employed to ascertain the minimal spectral and temporal data requirements to accurately and reliably monitor each assay in vivo or in vitro.

The detector is the imaging element from a commercially-available camera such as a CCD (charge-coupled device) from Andor Technology, having a holographic grating with 1800 groves/mm at 630 nm. The wavelength range for the grating is selected with a hand scan device (JY Inc.) and the housing for these parts is a SPEX 270M. Wavelength resolution is achieved with a 12 cm path length spectrograph. Spectral collection and interpretation may be performed on software installed in a computer. In field use the computer may be a portable laptop. To further minimize the size and power requirements, the optical train may be simplified by employing complementary metal oxide semiconductor (CMOS) type sensors for data collection and embedding simplified data control and analysis routines in the sensor electronics. The optical fiber at the sensing area of the SPR probe may be constructed of either silica or sapphire. The width of the fiber is preferably of submicron dimensions to about 200 microns. Both silica and sapphire fibers are biocompatible materials with silica being more flexible and sapphire being more durable. The refractive index (RI) range accessible to the SPR sensor is a function of the fiber tip material and geometry. The sensing region(s) are defined by removing the cladding from the fiber and depositing a 50 nm thick gold layer. A dextran layer between about 50 to 100 nm thick is then deposited on the gold. Antibodies to the antigens of interest are immobilized onto the dextran, creating a region that can sense antibody/antigen binding. This sensing zone will produce an SPR spectrum that is influenced by the binding of the antibodies and the refractive index of the biological fluid into which the probe is immersed. Sensing zones with the gold coat and the dextran, but without the antibodies, provide an SPR spectrum that is influenced only by the refractive index of the biological fluid into which the probe is immersed. The signal due to antibody binding can then be extracted as the difference between these two spectra.

Sapphire fibers can support a much thicker dextran/antibody/antigen layer than can silica fibers. The advantage of the thicker layer is the increased number of antibody bonding sites within the detection volume of the probe, which should lead to lower detection limits and greater dynamic range of the sensor. Also, the thick dextran hydrogel should partially shield the detection volume from RI changes due to fouling, nonspecific binding on the hydrogel surface, and optical density changes of the blood matrix.

To make the probe surface, the cladding is removed from the last 1 cm of the fiber nearest the tip. A 2 nm layer of chromium is sputter coated onto the bare fiber tip. The chromium layer has little effect on the SPR spectra, but is essential for ensuring the adhesion of the subsequent 50 nm gold layer. The gold layer supports the resonating surface plasmon. The optical properties of the surface plasmon changes with the RI of solution within 200 nm of the sensor. To achieve the necessary sensitivity and selectivity of RI changes, a layer of antibody-fixated dextran is attached via thiol linkage to the gold surface.

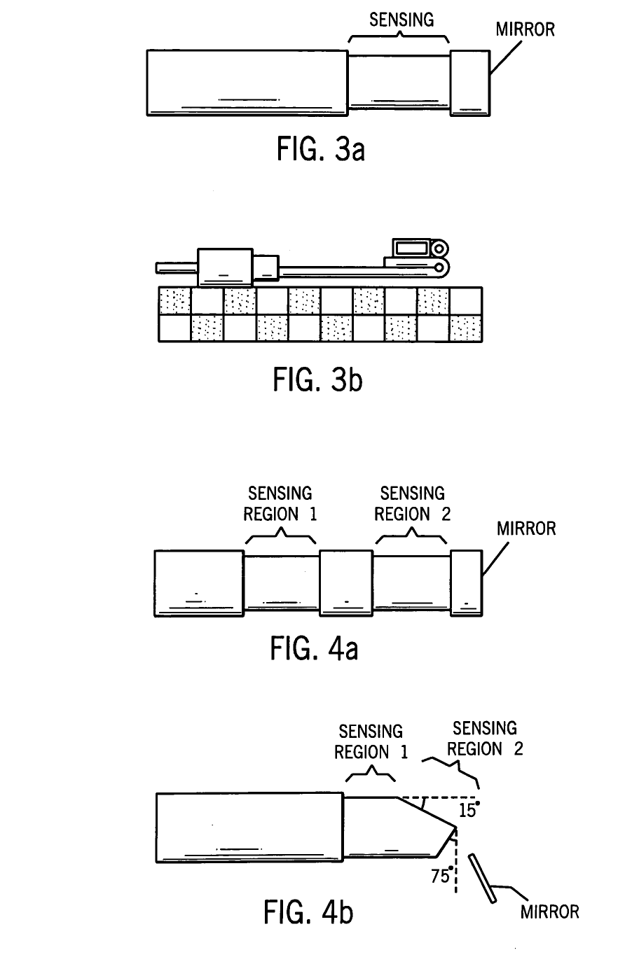

To prepare the probe surface, the optical fiber cladding and buffer are removed from the fiber to expose a ˜5 mm sensing area. A portion of the buffer is returned to protect the tip of the fiber during use (FIG. 3(a)). Multiple sensing areas can be incorporated in this manner. It is thus possible to employ one sensing area as a reference and the other sensing area as a sampling surface. The sampling surface is coated with an affinity-based reactive film specific for the analyte to be studied. In the case of the myocardial infarction sensors, the affinity-based film is comprised of immobilized antibodies specific to myoglobin, CRP, CK-MB, or cTnT or cTnI. Signals from both surfaces may be received and analyzed simultaneously for real-time in situ analysis.

A digital photograph of a short fiber optic SPR probe is presented in FIG. 3b. In this embodiment, the probe is terminated with a SMT type fiber optic connector for easy attachment and detachment to the sensor system.

Self Referencing SPR Sensors

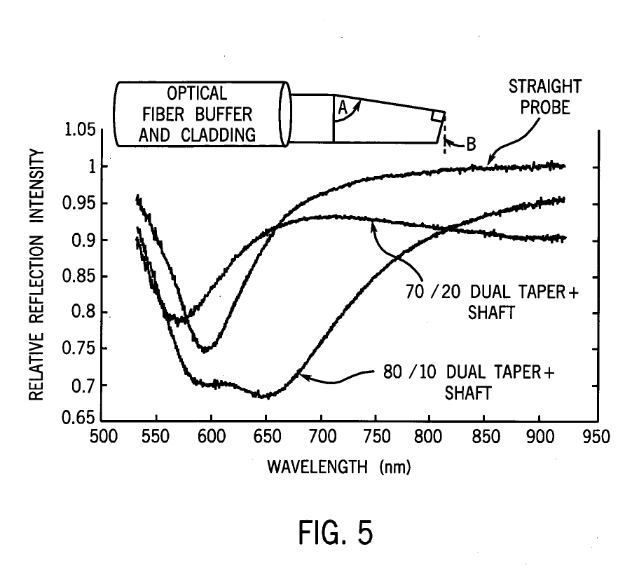

Fiber optic SPR sensors with two sensing zones have the potential to minimize the impact that nonspecific binding or bulk sample refractive index changes have on sensor performance. Without a reference probe in solution, it is impossible to determine if an observed change in RI is actually the result of target antibody-antigen interactions, fouling of the sensor surface, or a bulk matrix effect derived from temperature fluctuations. When a reference sensor is employed, it is assumed that any nonspecific binding, or bulk effects, will influence both sensors identically; thus the difference in signal between the two sensors is directly attributable to the target analyte. However, employment of two separate fiber optic sensors would significantly increase the size of the probe. An alternative is to construct self-referencing probes by putting both sensing zones on the same optical fiber. In a preferred embodiment of the invention, two-sensing-zone, self-referencing fiber optic probes are provided. In a first embodiment, two spatially separated sections of the cladding and buffer are removed from the optical fiber. This embodiment is shown in FIG. 4(a). The two separate sensing areas can be differentially treated during the antibody binding process. If the antibodies are left off of one sensing area or are rendered nonreactive to the antigens, one area responds to environmental changes only (non-specific binding) while the other area responds to environmental changes and antigen concentration. Multivariate calibration methods employ these two spectral sources of information. In other preferred embodiments, the tip of the fiber is beveled at complimentary angles (FIG. 4(b)). Beveling the fiber red shifts the SPR spectra for the beveled sensing area. This effect is illustrated in FIG. 5. The lesser (bluer) wavelength minimum in the reflectance spectra changes with the RI at the non-tapered part of the fiber probe, while the greater (redder) wavelength dip changes with the RI at the beveled tip of the probe. The degree of tapering determines the separation between these two dips. The advantage of the beveled probe is that a greater degree of wavelength separation between the active and reference sensing areas is achieved. With the straight probe, spectral separation is only achieved based on the RI difference derived from the presence of the antibodies. With the beveled probe, this spectral separation is also enhanced by the natural red-shift of the beveled region.

In an important aspect of the present invention a housing is provided to prevent fouling of the probe tip surface. The housing is located around the surface of the biosensor probe tip and shields the SPR sensor from cellular interference. The housing comprises one or more channels through which fluid can flow, but cells and other suspended particles cannot pass because of their size. Thus the target analyte can readily pass through the channels and bind with specific receptors on the sensor, but cells cannot pass through the channels and interact with the sensor. In those embodiments comprising competitive immunoassays, synthetic analyte molecules can be covalently bound to large nonreactive molecules and trapped inside the housing. These entrapped molecules will be able to compete with blood-borne analytes for binding with the receptors on the sensor. The housing is about 100 μm per side, small enough to fit into the implanting device, generally a catheter, but large enough to fit around the fiber optic sensor.

The sensor is coated with a low-bioactivity material to minimize cell-sensor and protein-sensor interactions. This biocompatible coating permits long useful lifetimes for the implanted sensors.

Construction of the Sensor Housing

The sensitivity of the present technique is diminished by non-specific binding to the reactive probe surface. Although the present biosensor achieves specificity by selection of a highly specific binding member to the analyte of interest, non-specific binding raises the background signal and reduces the sensitivity of the assays. Suspended particulate components are a major source of non-specific binding. Blood, for example, contains red and white blood cells that cause interference.

Nearly all implanted biomedical devices and materials are ultimately rejected by the body. To minimize unfavorable interactions with the body and maximize the sensor lifetime in the body, the sensor housing and sensing regions on the optical fibers are coated with low bioactivity materials. The sensor housing is made from polydimethyl siloxane (PDMS), which itself is a low-bioactivity polymer. It is functionalized and coated with oxidized dextran, which renders the surface highly biocompatible, so that fouling, nonspecific protein interactions, and initiation of an inflammatory response on the sensor housing can be eliminated or minimized. Surface treatment with Parylene-C brand para-chloro-xylylene (Specialty Coating Systems, Indianapolis, Ind.) also renders a low-bioactive surface. The parylene can also be coated with low bioactivity polymers or sugars or other molecules to further improve biocompatibility. A gold coating can be applied directly to the housing, and low-bioactivity polymers, molecules or sugars can be affixed to this coating. Many other polymers and surface coatings, such as heparin, can be applied in or on the housing to minimize fouling, nonspecific binding, the initiation of an inflammatory response and to improve the sensor lifetime in the body. The sensing region itself also is subject to fouling, nonspecific binding, and can be a catalyst for immune response. The dextran coating on the gold sensing region of the optical fiber acts to minimize these effects. Many other materials, such as polyethylene glycol, can be used to minimize non-specific binding at the sensor site. These surface treatments are examples of how the implanted lifetime of the sensor can be increased from roughly two to three weeks to time frames on the order of months. Chemical treatments of the housing and the dextran or other polymer or sugar of biocompatible molecule region on the sensor itself can also be used to extend the implanted lifetime. Oxidized surfaces and surfaces with plasma treatments that improve the hydrophilicity of the surface can be applied to minimize rejection.

Accordingly, a housing is provided to protect the reactive probe tip of the biosensor from contact with particulate components encountered in a fluid matrix in situ. The housing is designed with small holes to prevent the passage of larger particles to the probe surface, but to allow passage of soluble components and specifically the analyte of interest.

The housing comprises an elastomer, preferably polydimethylsiloxane (PDMS) a two-part elastomer from Dow Chemical Company. The housing is formed by photolithography on a photoresist template having a repeated pattern of posts of suitable dimensions. When the elastomer is released from the template, a film having fine holes results. The conformation of the film is modified to form a housing around the probe tip by treating the film with radio frequency (rf) oxygen plasma to allow the irreversible attachment of two plasma treated surfaces.

### Experimental Section

Procedure for Fabrication of Housing

The housing is produced by the following processes:

1. Fabricate photolithography masks

2. Fabricate SU-8 photoresist template

3. Treat template for PDMS mesh release

4. Spin PDMS mesh

5. Treat PDMS surface to improve biocompatibility

6. Form PDMS mesh into suitable conformation and attach to optical fiber probe.

Fabrication of Photolithography Mask

Patterning of materials using photolithography requires a mask. For feature sizes down to ˜50 μm, a mask generated in Adobe Illustrator and printed at a printer resolution of 5080 dpi is provided. Chrome masks such as are generally used in standard IC fabrication, may be used to produce feature sizes at the submicron level. For feature sizes between 50 μm and 10 μm, a glass emulsion mask is required. For feature sizes below 10 μm a chrome mask is required. In the present housing for screening small particles in biological fluids and tissues, feature sizes are optimally about 5 μm. Red blood cells, for example are 5-10 microns in diameter. It is possible using modern photolithography to make sub-micron-sized holes.

Fabricate SU-8 Photoresist Template

A bare Si wafer is cleaved into coupons that are about 1 inch × inch square. Hexamethyldisilizane (HMDS), an adhesion promoter, is spun at 4000 rpm for about 30 seconds. SU-8, a commercially available thick film photoresist (Microchem, Newton, Mass.), generally used in microfluidic device fabrication, is provided. SU-8 is spun at 1000 rpm for about 30 seconds to produce a film that is about 20 μm thick. The wafer is baked for about three minutes at 65° C. and then baked for about 1 hour at 95° C. After cooling, the wafer is exposed on a Karl Suss aligner, using a transparency mask, for 30 seconds at an intensity of 130 mW/cm³. The wafer is again baked for about three minutes at 65° C. and then baked for about 1 hour at 95° C. The construct is developed in SU-8 developer for about 30 seconds and rinsed in isopropanol until white residue appears. The development and rinsing step are repeated until no more white residue appears. The construct is then baked for one hour at 200° C.

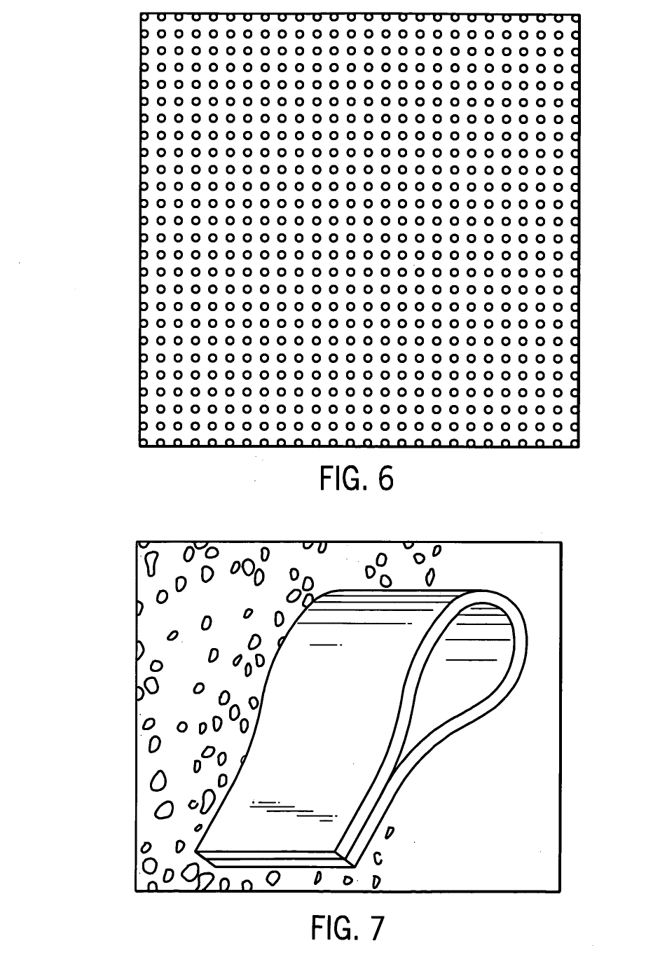

FIG. 6 is an SEM (Scanning Electron Microscope) image (19× magnification) of a template made from SU-8 photoresist on a silicon wafer using photolithography. The posts are ˜25 microns tall and ˜80 microns in diameter. A transparency (printed at 5080 dpi) was used as the mask for the fabrication of this template, but emulsion or chrome masks could be used to achieve feature sizes down to 10 or 5 microns respectively.

Treat Template for PDMS Mesh Release

The silicon surface is coated to prevent sticking of PDMS to bare silicon before spinning on the film. Preferably, the surface is coated with gold, but fluorination and chromium coating may also be applied [31, 32].

Spin PDMS Mesh

Structures of suitable dimensions are fabricated in PDMS by spinning it onto a template, curing, generally at about 1000° C. for 15 minutes, and peeling it off. These methods have been disclosed by Jackman (Jackman, R. J. et al., “Using Elastomeric Membranes as Dry Resists and for Dry Lift-Off: Langmuir, 1999. 15: 2971-2984.), herein incorporated by reference. It is an essential aspect of this step in this process to maintain a thickness of the PDMS film that is thinner than the photoresist posts that create the holes in the film.

Treat PDMS Mesh to Improve Biocompatibility.

The PDMS mesh is treated to make it biocompatible and specifically to prevent reactions with tissue in vivo. Preferably, the polymers are treated with ammonia plasma. The primary amine groups that are produced as a result of this treatment serve as attachment sites for oxidized dextran [33].

Coating with Dextran Improves Biocompatibility.

Chemical modification of the housing surface with parylene coatings may be used to prevent cells from attaching to the sensor housing and plugging the fluid flow ports. When parylene is the sensor coating, chemical modification and subsequent grafting of non-bioactive species to the parylene is desired. Using a remote microwave oxygen plasma or UV irradiation, the formation of surface aldehydes and carboxylic acid groups on parylene has been induced [34]. These species may be used for grafting low-bioactivity species onto the parylene. An alternative technology for providing a low-bioactivity surface on the housing includes the deposition of a gold layer with subsequent use of thiol-linkage technology to bind target low-bioactive molecules to the surface. Other inherently non-bioactive species also may be considered. These may or may not require additional processing to eliminate cell-housing interactions.

Attach Housing to Optical Fiber

The PDMS mesh is treated with a radiofrequency (rf) oxygen plasma causing the formation of —OH groups, which react with each other upon contact. This phenomenon may be used to cause the irreversible attachment of two plasma treated PDMS surfaces. The PDMS mesh may be attached to the optical fiber by modifying the conformation of the mesh using plasma treatment. For example, rings of PDMS may be painted on the optical fiber and then treated with plasma. These treated rings will be contacted with the treated surface of a PDMS mesh, resulting in attachment of the housing around the fiber. Alternatively, opposite surfaces of a PDMS mesh may be treated, the mesh wrapped around the fiber, and the treated surfaces brought into contact, causing them to adhere to each other to form a housing around the probe surface.

FIG. 7 is an SEM image of a polydimethylsiloxane (PDMS) film (16× magnification) that has adhered to itself due to treatment with a radiofrequency (rf) oxygen plasma. The treatment conditions were 50 sccm of O₂ at a pressure of 120 mtorr for 10 s at a power of 70 W. Upon contact the edges of the film adhered to each other irreversibly.

Affinity-Based Assays with the SPR Probe

In the present invention, conventional fiber-optic based SPR sensors have been modified by coating the probe tip with a film comprising a ligand having affinity for the analyte of interest. The miniaturized biosensor may be implanted in the tissue of an individual, by means of a catheter, e.g., where it generates signals concerning a biological marker in situ. Combining the sensitivity of SPR analysis with the selectivity of antibodies or other specific receptors yields a powerful sensor system. The probe tip comprises immobilized molecules capable of selectively binding to the target biological molecules at the tissue site. The immobilized molecule and the target marker molecule make up a binding pair.

In the present affinity-based SPR biosensor, traditional binding assays, such as competitive and sandwich Elisa methods, may be employed without using labeled molecules traditionally used in standard Elisa systems. It thus extends the use of affinity technology to in situ analyses. Its usefulness is as an affinity biosensor that allows real-time continuous analysis of biospecific interactions.

Preparation of the Optical Probe Surface for Affinity Assays

A fiber optic sensor similar to the one pictured in FIG. 3b was prepared by stripping the cladding and buffer from a 5 mm length of the fiber. The fiber was cleaned in a ‘piranha solution’ of hot 30% hydrogen peroxide and sulfuric acid to remove any oil and grease from the surface. The fiber was then sputter coated with 2 nm chromium and 50 nm gold.

A dextran layer was bound to the gold surface to provide a support for attaching the anti-myoglobin antibodies and prevent nonspecific binding of the myoglobin (or other proteins) to the sensor surface. A self assembled monolayer (SAM) of 11-mercapto-dodecanol was deposited on the gold surface by immersion in a 5 milimolar solution. This monolayer is then reacted with a 0.6M solution of epichlorhydrim in 5% diglyne and 50% Na OH 0.4M. Dextran T500 was then covalently bound to the alcohol end of the SAM. The hydroxyl groups on the dextran were carboxylated with bromoacetic acid and then activated with a mixture of EDC/NHS (N-ethyl-N′-(3-dimethylaminopropyl) carbodiimide HCl/N-hydroxysuccinimide). This produces reactive N-hydroxysuccinimide esters on the dextran layer and readies the sensor surface for a variety of antibody immobilization chemistries. While many attachment chemistries may be used, we have used the amine coupling method to immobilize the anti-myoglobin to the functionalized dextran layer.

In the case where it is needed to eliminate non-specific protein binding to the sensor, thiol-terminated polyethylene glycol (PEG) or other materials may be used to decorate the surface of the fiber optic sensor. PEG has been approved by the Food and Drug Administration for implantation in the human body. PEG molecules which are tethered to surfaces and exposed to an aqueous environment are highly hydrated and exhibit a large excluded volume. This property allows PEG to inhibit protein adsorption to surfaces by preventing dissolved proteins from approaching the surfaces closely enough to adhere. Methoxy-PEG-thiol is commercially available from Fluka Fine Chemicals. It has a molecular weight of 5000. This polymer can be bound to the gold surface of the optical fiber through a gold-thiol bond. It can also be coupled to the dextran. In the scheme, immobilized PEG surrounding the sensor will prevent nonspecific interactions with the surface while allowing specific receptor-ligand interactions.

The presence of the immobilized PEG or another coating prevents nonspecific protein binding to the surface, but it also influences the SPR signal. As discussed above, the SPR signal is determined by the change in molecular weight of surface bound species in the presence or absence of attachment of the target analyte.

In an important aspect of the present invention, methods are given for real-time measurement of biological molecules in vivo. In the method, molecules that are biological markers of clinical significance may be continuously monitored to track the progress of a disease or effectiveness of a therapy.

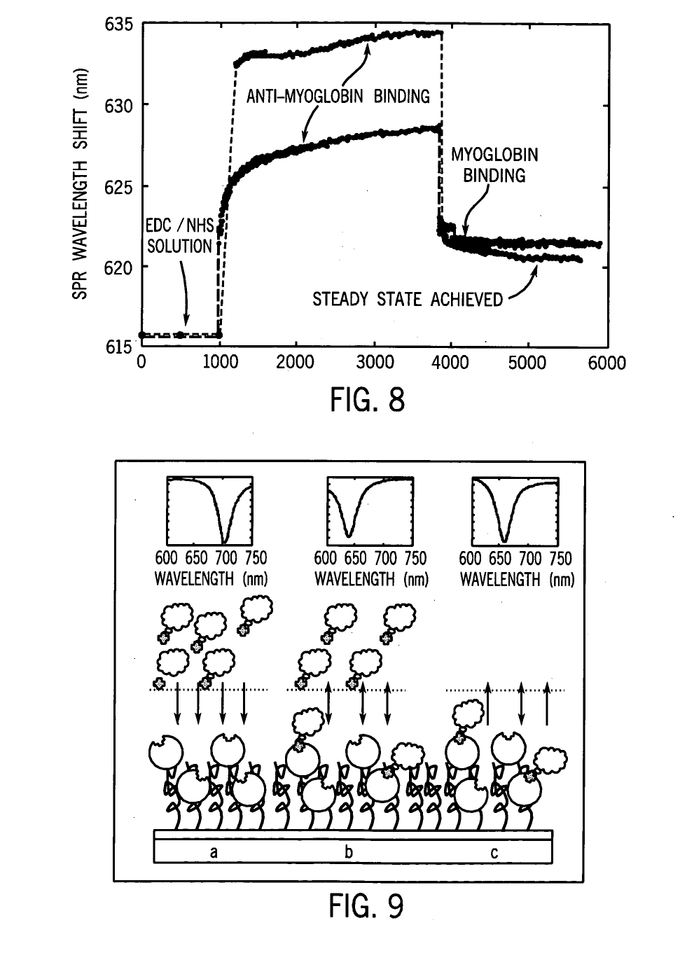

FIG. 8 is an illustration of the method of the present invention using the fiber optic SPR biosensor for in situ monitoring of human myoglobin. Myoglobin is one member of a binding pair and is the biological molecule of interest in this illustration. The other member of the binding pair is anti-myoglobin which is immobilized on the probe surface of the biosensor. In operation, a RI signal is received from the probe surface after it is placed in a tissue/biological fluid matrix and resides in situ. The SPR wavelength shift (nanometer) is measured over a period of time. Binding of myoglobin to immobilized anti-myoglobin is indicated in an increased shift after a period of time. Eventually a steady-state is achieved wherein no further binding occurs.

The method may be used to determine any biological molecule in vivo or in vitro that is one member of a binding pair when the other member of the binding pair is immobilized on the probe tip surface. Preferably the binding pairs are members of the group comprising antigen and antibody binding pairs wherein the antigen is a protein, peptide, carbohydrate, drug or other chemical compound, nucleotide and anti-nucleotide binding pairs, enzyme and receptor binding pairs, carbohydrate and lectin binding pairs, and pharmacological analytes and polymer binding pairs. Most preferably the binding pairs are antigen and antibody binding pairs and the antigen is selected from the group comprising protein, peptide, carbohydrate, drug or other chemical compound and the antibody is capable of bind specifically and with high affinity to the antigen.

The larger the molecular weight of the analyte, the more significant the change in the SPR signal when it binds to the immobilized binding member. FIG. 9 is a schematic illustration of the reactions that generate signals from the sensor. The dextran coated, antibody immobilized sensor exhibits an SPR spectrum that is a function of the surface coverage and thickness of dextran and antibody layer (FIG. 9a). Upon exposure to a concentration of target protein, a fraction of the proteins will bind to the antibodies based on the affinity constant for the binding pair. This will change the surface properties of the SPR sensor and a shift in the SPR spectrum, proportional to the antigen concentration, will be observed, as shown in FIG. 9b. Once the population of free antigens is removed, i.e., after a cardiac event, the bound antigen will partition off of the sensor surface. Then a regression of the SPR spectrum to its original form will be observed, as shown in FIG. 9c.

The molecular weight of the analyte plays an important role in the sensing. Cardiac myoglobin has a molecular weight of 17600 daltons (d); CK-MB=86,000 d; cTnT=33,000 d; and CRP ˜150,000 d. The larger molecular weight of the analyte, the more significant the change in the SPR signal when it binds to the selective receptor. These target molecules are of sufficient molecular weight to generate a detectable SPR shift upon binding with the sensor surface without signal amplification.

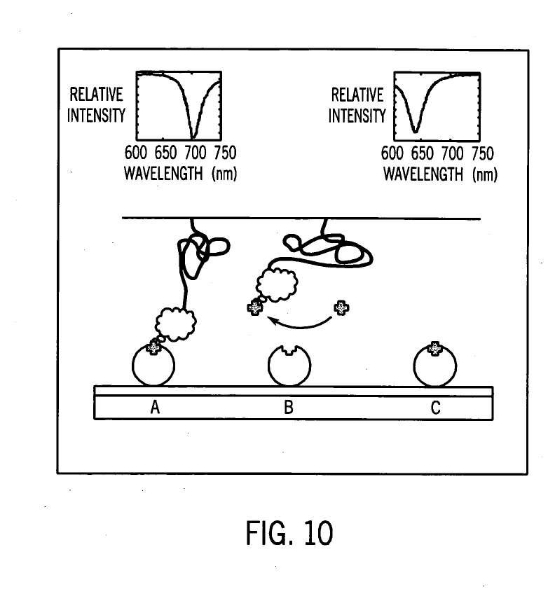

In the cases where target analytes may be too small to induce a significant SPR signal upon binding, signal amplification is needed. To increase the signal strength of low molecular weight analytes, a competitive binding assay strategy is proposed. As discussed below, the sensor is contained in a protective housing. Bovine serum albumin (BSA) or another high molecular weight molecule is anchored to the inside of the center and the free end of the molecule is to be tethered to the target analytes (i.e., cTnT, CRP, CK-MB and myoglobin). The BSA-analyte conjugate will never leave the sensor housing, but will compete with blood-borne analytes for binding, on the sensor. The BSA-tagged analyte is of sufficient molecular weight to elicit a red shift in the SPR spectrum relative to surface bound receptors alone. The presence of the BSA tag slightly hinders the association between the analyte and the receptor, therefore association of the receptor with the free analyte is thermodynamically favorable. Once the free analyte displaces the BSA-tagged analyte, the SPR spectral dip is blue shifted toward the SPR spectrum of unassociated bioreceptor, as shown in FIG. 10.

In preferred embodiments of the invention for determining myocardial infarction, the biological markers are selected from the group comprising cardiac troponin T (cTnT) or cardiac troponin I (cTnI), C-reactive protein (CRP), creatinine kinase myocardial band (CK-MB), and cardiac myoglobin (myoglobin). The cTnT, cTnI, CK-MB, and myoglobin are markers of cardiac cell death, while CRP is a non-specific acute phase reactant associated with higher risk of cardiac events in patients proteins which are released into the circulation during myocardial cell damage. CK-MB has been used historically to estimate the magnitude of infarctions. Myoglobin, a small protein, is rapidly released from damaged myocardial cells, often within 45 minutes after damage. Several classes of high-risk patients who experience silent infarction, such as diabetics or dialysis patients, will benefit from this sensor as it tells them when they have had an event so that they may seek treatment. These sensors also should be useful in monitoring at-risk patients exhibiting conflicting symptoms in an emergency room, ambulatory, hospital, or remote/field setting.

The sensors may be used, as well, and in similar manner, to detect other medical problems including measurement of brain CK-BB to detect strokes, CRP to detect tissue rejection and additional problems, including death or disease of cells and tissues, disease progression, and metabolic changes, or even concentrations of poisons and other foreign substances. The sensors may also be employed for in vivo ‘ligand fishing’ to detect and capture currently unknown or unrecognized analytes that may be of future diagnostic value.

These markers are a first member of a binding pair and the second member of the pair, antibodies specific for these markers, are immobilized on the probe tip surface of the biosensor. In the method, the probe comprising the specific antibodies is implanted into an individual and SPR wavelength shift is observed over a period of time. The shift is the result of binding between the pairs on the surface, which changes the RI signal of the probe. To calibrate the shift, and to make the method quantitative, the results are compared to measurements obtained from a similar tissue having a known amount of myoglobin.

In other preferred embodiments of the invention a method is provided for using the biosensor for monitoring the progression of wound healing in a tissue in an individual to distinguish between healing and non-healing wounds. It is often a problem in burn victims that a wound being observed superficially seems to be healing but is nevertheless progressing negatively. The healing trajectory for a successful outcome involves platelets and clotting (homeostasis). The biochemical response works through an inflammatory phase (about one week), a proliferative phase (weeks or months) and a maturation phase (months to years). In many cases, about 5%, healing does not proceed through these phases. It has been observed in these non-healing cases that inflammatory chemical signalers such as interleukin 1 and interleukin 6 are in higher concentration than in healing wounds. Matrix proteolases, biological markers of tissue breakdown, are also in higher concentration.

Indicators of wound non-healing allow physician intervention to ameliorate the condition. Burns by fire are common occurrences in our cities. Treatment of bums has become a subspecialty in medicine today, with key hospitals in larger cities now having specialized burn centers. Recent methods for treating burns have decreased mortality and morbidity and have shortened hospital stay and costs. However, treatment of patients with burns over the age of 80 still has made no difference in mortality.

Studies over the past few years have indicated that there are specific factors in both burn fluids and in tissue healing exudates, which modify rates of healing. Attention has been focused on the interleukins, especially IL-1, IL-4 and IL-6, and the tumor necrosis factor-alpha (TNF-alpha). The latter delays wound and burn healing, while the interleukins either delay or accelerate healing.

In these methods, the biological marker is selected from the group comprising interleukins, matrix proteolases and the tumor necrosis factor-alpha (TN-F-alpha). An antibody capable of binding specifically and with high affinity to the biological marker is immobilized on the SPR probe tip surface. The shift in RI signal received after the probe is implanted into the wound is observed and correlated with concentration of the biological markers of wound non-healing. The biosensor coated with antibodies to the above markers to measure their concentrations in exudates collected from bums and wounds. These in vitro measurements are being supplemented by in situ measurements, where the biosensor is placed directly over the burn or wound healing area under the surgical dressings. Once changes in the concentration of these markers are detected, appropriate treatments can be started.

In other preferred methods of the present invention the detection of breast cancer is presented. Cancer of the breast is the most common form of cancer in women. About 10% of all women develop breast cancer during their lives. She may be of increased risk if she has a family history of the disease, if she has had her first child after age 30, if she has begun menstruating early, or if she has been on hormonal replacement therapy after menopause. The common clinical diagnostic tests include mammography, but many women do not avail themselves of this test and mammography is less effective in detecting tumors in younger patients under age 50.

Confirmed breast cancer patients release markers CA 15-3 and CA 27-29. Monitoring of blood in women patients for this markers would permit screening of large numbers of women in risk for breast cancer. In the method of the present invention, the SPR optical probe tip will be coated with antibodies specific for these markers. Assays using the immobilized antibodies permit both in vivo and in vitro monitoring of blood for these early markers of disease. The miniaturized system designed for field use will be especially useful for bringing these assays to patients outside large urban areas where more sophisticated assays are available.

Occasionally, post-operative patients still show elevated levels of these markers, which often is a sign of recurrence of the breast tumor.

With the SPR biosensors of the present invention it is possible to measure these markers either in vitro in a blood sample from the patient, or in vivo by introducing the probe into any vein.

Methods of the present invention may be used to monitor 1) women for possible recurrence of the tumor after surgery, 2) women without access to other means of detection, especially if mammography is not available or refused, 3) women with known risk factors such as family history or hormonal replacement therapy, and 4) women who want to optimize their chances for early detection.

A similar rationale is in the use of another tumor marker CA 125, which can indicate ovarian cancer. It is estimated that about 25,000 women will be diagnosed this year with ovarian cancer with about 15,000 deaths. This form of cancer is much more difficult to detect, and frequently women present themselves to physician's offices complaining of bone pain, due to metastasis of the ovarian tumor. Bimanual pelvic examinations, pelvic ultrasound examinations and surgical biopsy are diagnostic, since simpler tests are not easily available. CA 125 is detected in 80% of women with ovarian cancer. The present SPR biosensor coated with antibodies to CA 125 will be a simpler way to detect this ovarian cancer marker. The biosensor may be used both in vitro and in vivo to screen women at high risk for this type of cancer, which includes women with a positive family history.

Other medical detection uses for the described probe include, but are not limited to, drug level detection, biological warfare detection and pesticide detection.

In other preferred embodiments of the present invention, the implanted biosensors may be used as part of an integrated drug delivery system. In these embodiments, the sensor output is passed to an integrated microchip that performs signal reduction processes and determines the levels of target drugs, hormones, or other biochemical species in the blood or other biological fluids or tissues. The microchip can then be used to direct the delivery of drugs, proteins, hormones, or other therapeutic agents directly into the body through the use of an integrated, implanted pump and reservoir system or controlled therapy release agent. This application of the sensor would provide great advantages to diabetics, by monitoring glucose and insulin levels in the bloodstream and regulating insulin delivery automatically rather than causing the patient to draw and test his or her own blood and administer a shot of insulin. This would result in a much more responsive and even level of therapy delivery, for better disease management. Similar approaches can be taken to treat other hormone-based diseases, including but not limited to hypothyroidism, underproduction of estrogen and progestin in post-menopausal women as well as non-hormone based conditions that require monitoring and therapy delivery over time.

### Experimental Details

### EXAMPLE 1

This example illustrates the method of the present invention for analysis of human myoglobin in blood.

Anti-myoglobin (human myoglobin antiserum) and human myoglobin positive control (from ICN Pharmaceuticals) in lyophilized powder form were reconstituted with deionized water. The optical probe surface was rinsed with a mixture of HEPES, NaCl, EDTA, and Surfactant P20 at pH 7.0 (HBS) to condition the sensor surface. Once the dextran layer was activated with the EDC/NHS solution as described hereinabove, the anti-myoglobin was immobilized by dipping the sensor into a ppm solution of reconstituted antiserum. Tracking the minima of the SPR spectra showed binding of the antibody. For the particular sensor employed in constructing FIG. 8, the EDC/NHS activated probe yielded a minimum in the reflected spectrum at approximately 616.5 nm and was stable over time. The surface plasmon resonance minimum shifted to higher wavelengths as the anti-myoglobin bound to the activated dextran. The anti-myoglobin was allowed to react with the activated dextran for 50 minutes although the immobilization of the anti-myoglobin to the dextran was largely complete after 15 minutes.

Following immobilization of the anti-myoglobin, the sensors were rinsed with 1 M ethanolamine hydrochloride to deactivate excess esters and desalt loosely bound antibodies. The positive control (myoglobin) was then bound to the antiserum by dipping the sensor in a ˜2 ng/ml solution of myoglobin. FIG. 8 shows initial sensing of myoglobin in less than 1 minute, with approximately 10 minutes required before the concentration of bound myoglobin reached steady state. Note that calibration of the sensor does not requite steady state analysis. The initial rate of myoglobin binding to the sensor is proportional to the concentration of myoglobin in the solution. Thus initial estimates of myoglobin concentration can be made from the rate of change in the SPR signal. It has been observed that when the sensor is removed from the myoglobin-rich solution, myoglobin is rapidly released from the antibodies, with a corresponding reduction in the SPR signal. This indicates that the binding is reversible, so that the sensor can be used to track increases and reductions in cardiac damage marker levels in the bloodstream.

### EXAMPLE 2

This example illustrates the method of the present invention for measuring biological markers of myocardial infarction in vivo on a continuous real-time basis.

Biological markers of myocardial infarction comprise cardiac troponin T (cTnT), C-reactive protein (CRP), creatinine kinase, myocardial band (CK-MB), and cardiac myoglobin (myoglobin).

Antibodies to these markers are immobilized on a probe surface of a multifiber optical sensor so that each fiber contains an antibody to one of the markers. The multfiber probe surface is inserted through a catheter into the vein of an individual suspected of undergoing myocardial infarction. A first signal is received by a spectrophotometer attached to the sensor. This signal represents the reflectance and the minimum refractive index of the probe and is the background signal received from the blood where the probe resides in situ. A second signal is recorded after a period of time at a shifted wavelength. This signal represents the reflectance and the minimum refractive index of the probe after reaction between the probe surface and the target marker. Differences between the two wavelengths are measured and the difference is compared to values from a model blood system having the target molecules at known concentration. After a period of time the probe surface and housing around the probe surface within the catheter are flushed with heparin to remove interfering substances formed in situ. A new background signal is recorded and measurements are repeated.

### EXAMPLE 3

The in situ continuous assay of Example 2 may be used to monitor therapy by measuring blood components that are biological markers caused to change in concentration during the course of therapy.

### EXAMPLE 4

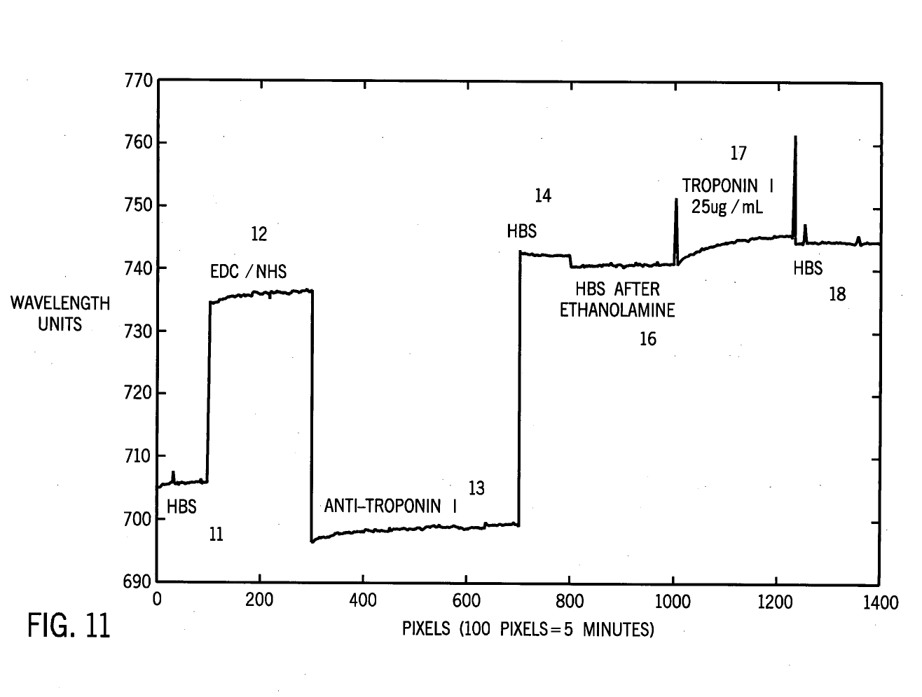

This example illustrates the method of the present invention for determining cardiac troponin I at low concentrations. The numbers in parentheses below refer to the numbers on the graph in FIG. 11.

Preparation of Probe

The fiber-optic probe was placed in HBS (10 mM HEPES, 3.4 mM EDTA and 0.00f % Tween 20 at pH 7.4) for 5 minutes (11). The probe was then placed in 1:1 solution of EDC (N-(3-dimethylaminopropyl)-N′-ethylcarbodiimide hydrochloride) 0.4M:NHS 0.01M (N-hydroxysuccinimide) (12). Next the probe was placed in a solution of anti-troponin I at a concentration of 500-700 mg/ml at pH=4 (13). The fiber optic probe was left in the antibody solution for 20 minutes. The probe was washed with HBS for 5 minutes (14). The probe was placed in a IM aqueous solution of ethanolamine at pH 8.4 (15). The buffer used for the pH=4 solution is 10 mM sodium acetate. The fiber-optic probe was washed in HBS for t minutes (16).

Reaction with Target Biomolecule—Troponin I

The probe was placed in a solution of Troponin I at a desired concentration in HBS for 20 minutes (17).

Regeneration of Probe

The probe was washed in HBS (18) and then regenerated by contact with 10 mM glycine pH=2 for 4 minutes (19).

### EXAMPLE 5

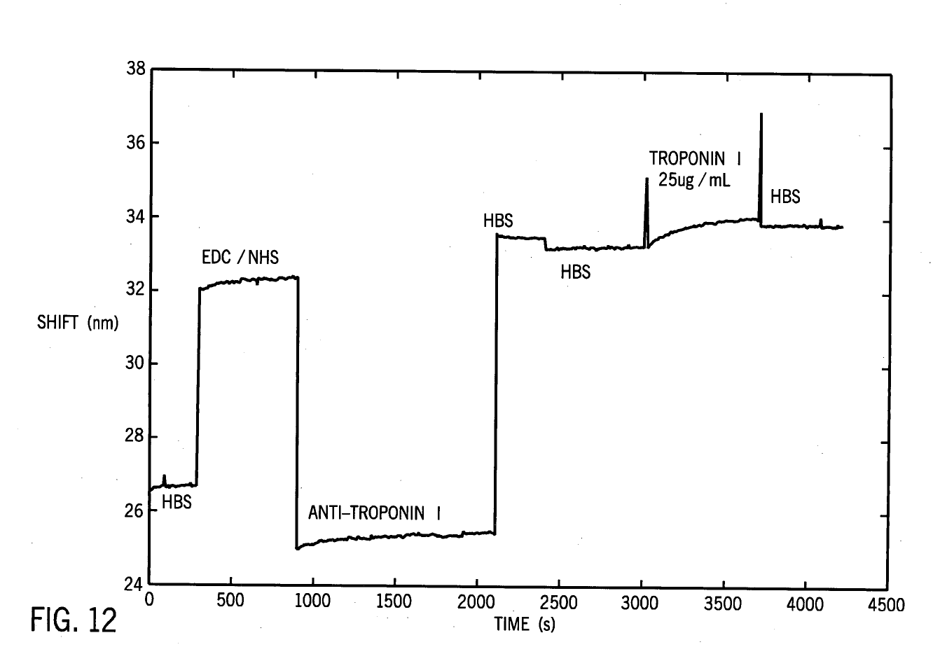

This example illustrates the detection of nanogram amounts of troponin I using the affinity-based SPR biosensor. The results of an assay of 25 microgram troponin I by the method of Example 3 is given in FIG. 12.

### EXAMPLE 6

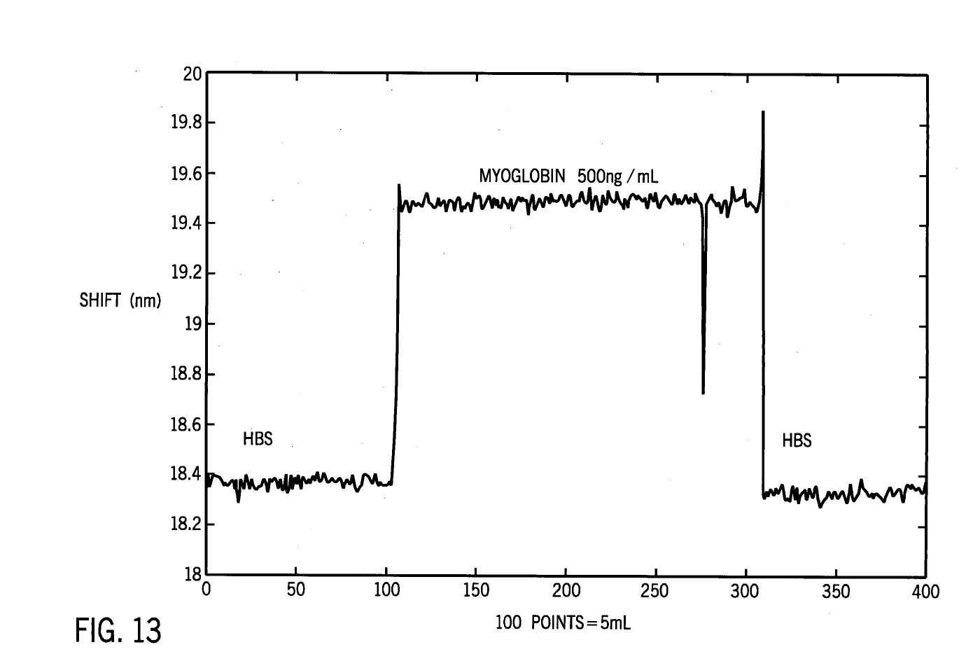

The results of assay of a 500 ng solution of myoglobin are given in FIG. 13.

### EXAMPLE 7

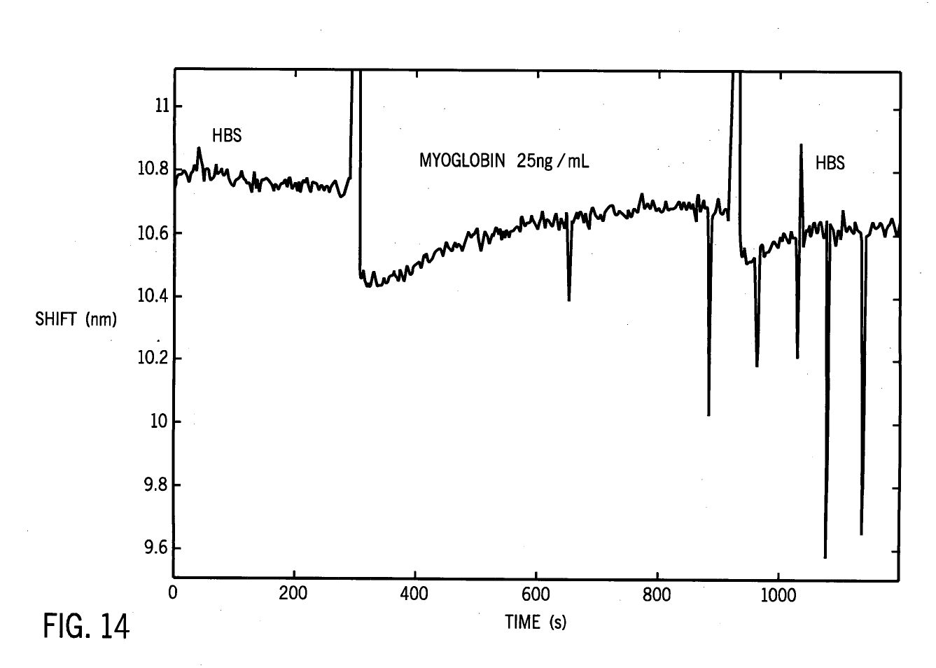

The results of assay of a 25 ng solution of myoglobin are given in FIG. 14.

### EXAMPLE 8

This example illustrates the method of using the biosensor of the present invention for the detection of breast cancer.

The method of Example 1 was repeated to determine the presence of biological markers CA15-3 and CA 27-29 in blood removed from a woman patient. Anti-CA15-3 and antiCA 27-29 were immobilized on the probe surface. Analysis of wavelength shift from the blood sample due to binding between immobilized antibody and marker were recorded to determine presence of the disease.

With our sensors we are able to measure these markers either in vitro in a blood sample from the patient, or in vivo by introducing the probe into any vein. These biosensors will be useful in monitoring 1) women for possible recurrence of the tumor after surgery, 2) women without access to other means of detection, especially if mammography is not available or refused, 3) women with known risk factors such as family history or hormonal replacement therapy, and 4) women who want to optimize their chances for early detection.

A similar rationale is in the use of another tumor marker CA 125, which can screen for ovarian cancer. It is estimated that about 25,000 women will be diagnosed this year with ovarian cancer with about 15,000 deaths. This form of cancer is much more difficult to detect, and frequently women present themselves to physician's offices complaining of bone pain, due to metastasis of the ovarian tumor. Bimanual pelvic examinations, pelvic ultrasound examinations and surgical biopsy are diagnostic, since simpler tests are not easily available. CA 125 is detected in 80% of women with ovarian cancer.

Our biosensor coated with antibodies to CA 125 will be a simpler way to detect this ovarian cancer marker. We are using the sensor both in vitro and in vivo to screen women at high risk for this type of cancer, which includes women with a positive family history.

Although certain preferred embodiments and methods have been disclosed herein, it will be apparent from the foregoing disclosure to those skilled in the art that variations and modifications of such embodiments and methods may be made without departing from the spirit and scope of the invention.

### References

1. A. Brecht and G. Gauglitz, Anal. Chem. Acta, 347, 1997, pp. 219-233.

2. M.-P. Marco, S. Gee, B. D. Hammock, TRAC, 14, 1995, pp. 341-350.

3. C. E. H. Berger, T. A. M. Beumer, R. P. H. Kooyman, and J. Greve, Anal. Chem. 70, 1998, pp. 703-706.

4. K. Alfthan, Biosensors and Bioelectronics, 13, 1998, pp. 653-663.

5. M. Knibiehler, F. Goubin, N. Escalas, Z. O. Jonsson, H. Maraguil, U. Hubscher, and B. Ducommun, FEBS Letters, 391, 1996, pp. 66-70.

6. R. Karlsson, Anne Michaelsson, and Lars Mattsson, J. Immun. Meth., 145, 1991, pp. 229-240.

7. C. E. Jordan and R. M. Corn, Anal. Chem., 69,1997, pp. 1449-1456.

8. J. Lerme, B. Palpant, B. Prevel, E. Cottancin, M. Pellerin, M. Treilleux, J. L. Vialle, A. Perez, and M. Broyer, Eur. Phys. J. D., 4, 1998, pp. 95-108.

9. H. E. de Brujin, R. P. H. Kooyman, and J. Greve, Applied Optics, 29, 1990, pp. 1974-1978.

10. W. J. Bender, R. E. Dessey, M. S. Miller, and R. O. Claus, Anal. Chem., 66, 1994, pp. 963-970.

11. E. G. Ruiz, I. Garces, C. Aldea, M. A. Lopez, J. Mateo, J. Alonso-Chamarro, and S. Alegret, Sensors and Actuators A, 37-38, 1993, pp. 221-225.

12. K. Matsubara, S. Kawata, and S. Minami, Appl. Spectrs., 42, 1988, pp. 1375-1379.

13. M. N. Weiss, S. Srivastava, and H. Groger, Electronics Letters, 32, 1996, pp. 842-843.

14. L. S. Jung, C. T. Campbell, T. M. Chinowsky, M. N., and S. S. Yee, Langmuir, 14, 1998, pp. 5636-5648.

15. M. Hausch, D. Beyer, W. Knoll, and R. Zentel, Langmuir, 14, 1998, pp. 7231-7216.

16. C. R. Lawrence, A. S. Martin, and R. Sambles, Thin Solid Films, 208, 1992, pp.269-273.

17. A. G. Frutos and R. M. Corm, Anal. Chem., 70, 1998, pp. 449A-455A.

18. S. Sasaki, R. Nagata, B. Hock, and I. Karube, Anal. Chem. Acta, 368, 1998, pp. 71-76.

19. S. Lofas and B. Johnsson, J. Chem. Soc., Chem. Commun., 1990, pp. 1526-1528.

20. S. Lofas, B. Johnsson, A. Edstrom, A. Hannson, G. Lindquist, R.-M. M. Hillgren, and L. Stigh, Biosensors and Bioelectronics, 10, 1995, pp. 813-822.

21. C. E. H. Berger, T. A. M. Beumer, R. P. H. Kooyman, and J. Greve, Anal. Chem., 70, 1998, pp. 703-706.

22. J. Homola, G. Schwotzer, H. Lehman, R. Willsch, W. Ecke, H. Bartelt, Chemical, Biochemical, and Environmental Fiber Sensors VII, 2508, 1995, pp. 324-333.

23. K. S. Johnston, S. S. Yee, K. S. Booksh, Anal. Chem., 69, 1997, pp. 1844-1851.

24. M. K. Boysworth, IL A. Obando, K. S. Booksh, Proceedings of SPIE, 3856, R. Shaffer, R. Potyrailo, eds., 1999, pp. 308-316.

25. Duffy, D. C., J. C. McDonald, O. J. A. Schueller, and G. M. Whitesides, Analytical Chemistry, 70, 1998, pp. 4974-4984.

26. Duffy, D. C., O. J. A. Schueller, S. T. Brittain, and G. M. Whitesides, Journal of Micromechanics and Microengineering, 9(3), 1999, pp. 211-217.

27. Lee, L. J., M. J. Madou, K. W. Koelling, S. Daunert, S. Lai, C. G. Koh, Y. Juang, Y. Lu, and L. Yu, Biomedical Microdevices, 3(4), 2001, pp. 339-351.

28. McDonald, J. C., D. C. Duffy, J. R. Anderson, D. T. Chiu, H. Wu, O. J. A. Schueller, and G. M. Whitesides, Electrophoresis, 21, 2000, pp. 27-40.

29. Whitesides, G. M. and A. D. Stroock, Flexible Methods for Microfluidics. Physics Today, 54(6), 2001, pp. 42-50.

30. Jackman, R. J., D. C. Duffy, O. Cherniavskaya, and G. M. Whitesides, Langmuir, 15, 1999, pp. 2973-2984.

31. Jo, B. H., L. M. Van Lerberghe, K. M. Motsegood, and D. J. Beebe, Journal of Microelectromechanical Systems, 9(1), 2000, pp. 76-81.

32. Hong, S., Z. Tang, D. Djukic, A. Tucay, S. Bakhru, R. Osgood, J. Yardley, A. C. West, and V. Modi. in 2001 Microelectromechanical Systems Conference. 2002. Berkeley, Calif.

33. Dai, L., H. A. St. John, J. Bi, P. Zientek, R. C. Chatlier, and H. J. Griesser, Surface and Interface Analysis, 29, 2000, pp. 46-55.

34. Callahan, R., Raupp, G., and Beaudoin, S., Journal of Vacuum Science and Technology B, 19(3), 2000, pp. 725-731.

We claim:

1. An optical fiber surface plasmon resonance (SPR) biosensor for observation of a biological marker in a biological fluid wherein said biological marker is a first member of a binding pair, and said biosensor comprises:
a. an SPR probe surface having immobilized thereon a second member of said binding pair;
b. a spectrophotometric means for receiving a first signal from said probe surface and a second signal from said implanted probe surface, said second signal being received at a time after binding of said first and second members of said binding pair on said probe surface; and
c. means for comparing properties of said first received signal and said second received signal to determine the presence of said biological marker.

2. The biosensor of claim 1, wherein said optical fiber probe surface comprises dextran as immobilization agent for said first member of said binding pair.

3. The biosensor of claim 1, wherein said probe surface comprises polyethyleneglycol as anti-fouling agent.

4. The biosensor of claim 1, for in vivo observation of a biological marker in a tissue in an individual wherein said SPR probe surface is implantable in said individual and said biosensor comprises in addition a housing for said probe surface, said housing being capable of excluding particulate components of said fluid and thereby preventing fouling, inflammatory response and non-specific binding of said components to said probe surface.

5. A biocompatible screen for housing an implanted medial device in an individual, comprising an elastomeric screen formed by a lithographic process, said screen having penetrating holes of between 1 and 50 microns in diameter.

6. The screen of claim 5, wherein said medical device is a fiber optic biosensor or a catheter.

7. The biosensor of claim 2, wherein said SPR probe surface is implanted through a catheter into said individual.

8. The biosensor of claim 2, in which said optical fiber is of a width of less than 1 micron to about 200 microns.

9. The biosensor of claim 8, wherein the width of said optical fiber is about 10 microns to 100 microns.

10. The biosensor of claim 1, in which said binding pair is an antigen and antibody binding pair, nucleotide and anti-nucleotide binding pair, enzyme and receptor binding pair, carbohydrate and lectin binding pair, or a pharmacological analytes and polymer binding pair.

11. The biosensor of claim 10, in which said antigen is a protein peptide, carbohydrate, drug or other chemical compound and said antibody is capable of binding specifically and with high affinity to said antigen.

12. The biosensor of claim 1, in which said biological marker is a protein, peptide, RNA, DNA or carbohydrate.

13. The biosensor of claim 4, for detecting myocardial infarction in an individual in which the first member of said binding pair CtNT, CtnI, CRP, CK-MB or myoglobin and said second member of said binding pair is antibody capable of binding specifically to said first member.

14. The biosensor of claim 1, wherein said received signals are wavelengths of minimum reflectance.

15. The biosensor of claim 1, wherein said optical probe comprises multimode optical fibers.

16. The biosensor of claim 1, wherein said optical probe comprises self-referencing optical sensors.

17. The biosensor of claim 2, wherein said received signals are wavelengths of minimum reflectance.

18. The biosensor of claim 2, wherein said optical probe comprises multimode optical fibers.

19. The biosensor of claim 2, wherein said optical probe comprises self-referencing optical sensors.

20. The biosensor of claim 19, wherein said self-referencing optical sensor comprises a beveled tip.

21. The biosensor of claim 19, wherein said self-referencing optical sensor comprises spatially separated sensing areas.

22. The biosensor of claim 1, wherein said fluid is blood, cerebrospinal fluid, mucous membrane, wound tissue, implanted organs, nervous tissue and associated fluids or urine.

23. A system for detecting a biological molecule in a fluid comprising the biosensor of claim 1, and comprising in addition a spectrophotometer for determining the wavelength of minimum reflectance from each said probe surfaces and a means for calculating differences between said wavelengths.

24. An in vitro method for detecting a biological molecule in a tissue matrix and associated fluid from an individual comprising the steps of:
a. contacting the biosensor of claim 1, with said tissue or associated fluid;
b. spectrophotometrically receiving said first signal;
c. spectrophotometrically receiving said second signal;
d. calculating differences between said received signals; and
e. comparing said calculated differences to signals received from a standard tissue containing said biological molecule to determine the presence of said biological molecule.

25. An in vivo method for detecting a biological molecule in a tissue matrix and associated fluid in an individual comprising the steps of:
a. implanting the biosensor of claim 2, at a selected site in said tissue;
b. spectrophotometrically receiving said first signal;
c. spectrophotometrically receiving said second signal;
d. calculating differences between said received signals; and
e. comparing said calculated differences to signals received from a standard tissue containing said biological molecule to determine the presence of said biological molecule.

26. The method of claim 25, wherein said received signals are wavelengths of minimum reflectance.

27. The method of claim 25, wherein said tissue matrix and associated fluid is blood, urine, cerebrospinal fluid, mucous membrane, wound tissue, or implanted organs and associated fluids.

28. A method of claim 25, for detecting myocardial infarction in an individual wherein said biosensor comprises a probe surface having immobilized thereon antibodies capable of binding specifically to a member of the group comprising cardiac troponin T (cTnT), cardiac troponin I (cTnI), C-reactive protein (CRP), creatinine kinase, myocardial band (CK-MB), and cardiac myoglobin (myoglobin).

29. The method of claim 25, for continuous in vivo monitoring of said individual wherein said probe surface is inserted intravenously into said individual and measurements are repeated over a period of time.

30. The method of claim 25, for screening an individual for the presence of breast cancer wherein said biosensor comprises a probe surface having immobilized thereon antibodies capable of binding specifically to a member of the group comprising CA 15-3 and CA 27-29.

31. A method for quantifying the amount of a biological molecule in vivo in an individual comprising the method of claim 25, and comprising in addition the step of comparing the observed properties of said signals to signals received from a biological solution or tissue and associated fluid having a known concentration of said biological molecule.

32. The method of claim 25, for continuous in situ observation of said biological marker over a determined period of time wherein said biosensor is allowed to remain in situ and said signals are repeatedly received over said period of time.

33. The method of claim 32, for monitoring therapy of a medical condition wherein the presence of said biological marker changes over a period of time in response to said therapy.

34. The method of claim 33, comprising in addition a means for delivering a chemical agent to said in situ site in response to a signal from said biosensor.

35. A method of monitoring a human or animal subject for the presence or absence of a marker indicative of the presence or absence of a medical condition comprising:
a. providing an SPR probe having a first surface with a binding member adherent thereto, the binding member being effective to bind to the marker;
b. providing an optical path to the first surface;
c. locating the probe at a location of interest in or on the subject;
d. directing light to the first surface along the optical path;
e. observing the light retrieved from the probe for spectral indications of the presence or absence of the marker.

36. A method of monitoring according to claim 35, wherein step a) comprises providing a housing for the probe, including providing a filtering housing surface adapted to filter out particles to avoid interference by such particles in the spectral indication of the presence or absence of the marker.

37. A method of monitoring according to claim 35, wherein step b) comprises providing a catheter, the catheter comprising an optical fiber path, and step c) comprises intravenously moving the probe into place with the catheter.
